(19) United States
(12) Patent Application Publication
(10) Pub. No.: US 2014/0085101 A1
(43) Pub. Date: Mar. 27, 2014

(54) DEVICES AND METHODS TO FACILITATE AFFECTIVE FEEDBACK USING WEARABLE COMPUTING DEVICES

(71) Applicants: Hosain Sadequr Rahman, San Francisco, CA (US); William B. Gordon, Woodside, CA (US)

(72) Inventors: Hosain Sadequr Rahman, San Francisco, CA (US); William B. Gordon, Woodside, CA (US)

(73) Assignee: AliphCom, San Francisco, CA (US)

(21) Appl. No.: 13/831,301

(22) Filed: Mar. 14, 2013

Related U.S. Application Data
(60) Provisional application No. 61/705,598, filed on Sep. 25, 2012.

(51) Int. Cl. A61B 5/00

(52) U.S. Cl. CPC A61B 5/0002
USPC 340/870.01

(57) ABSTRACT

Various embodiments relate generally to electrical and electronic hardware, computer software, wired and wireless network communications, and computing devices, including mobile and wearable computing devices, and more specifically, to devices and techniques for assessing affective states of a user based on data derived from, for example, a wearable computing device. In one embodiment, an apparatus including a wearable housing configured to couple to a portion of a limb at its distal end, a subset of physiological sensors and a processor configured to execute instructions configured to calculate a portion of an intensity associated with an affective state for each of the physiological, form an intensity value based on the portions of the intensity and determine a polarity value of the intensity value. The apparatus is further configured to determine the affective state, for example, as a function of the intensity value and the polarity value of the intensity value.

### CROSS-RELATED APPLICATIONS

This application claims the benefit of U.S. Provisional Patent Application No. 61/705,598 filed on Sep. 25, 2012, which is incorporated by reference herein for all purposes.

### FIELD

The various embodiments of the invention relate generally to electrical and electronic hardware, computer software, wired and wireless network communications, and computing devices, including mobile and wearable computing devices, and more specifically, to devices and techniques for assessing affective states (e.g., emotion states or moods) of a user based on data derived from, for example, a wearable computing device.

### BACKGROUND

In the field of social media and content delivery devices, social networking websites and applications, email and other social interactive services provide users with some capabilities to express an emotional state (or at least some indications of feelings) with whom they are communicating or interacting. For example, Facebook® provides an ability to positively associate a user with something they like, with corresponding text entered to describe their feelings or emotions with more granularity. As another example, emoticons and other symbols, including abbreviations (e.g., LOL expressing laughter out loud), are used in emails and a text messages to convey an emotive state of mind.

While functional, the conventional techniques for conveying an emotive state are suboptimal as they are typically asynchronous—each person accesses electronic services at different times to interact with each other. Thus, such communications are usually not in real-time. Further, traditional electronic social interactive services typically do not provide sufficient mechanism to convey how one's actions or expressions alter or affect the emotive state of one or more other persons.

Thus, what is needed is a solution for overcoming the disadvantages of conventional devices and techniques for assessing affective states (e.g., emotion states, feelings or moods) of a user based on data derived using a wearable computing device.

### BRIEF DESCRIPTION OF THE DRAWINGS

Various embodiments or examples (“examples”) are disclosed in the following detailed description and the accompanying drawings:

FIG. 1 illustrates an exemplary system for assessing affective states of a user based on data derived from, for example, a wearable computing device, according to sonic embodiments;

FIG. 2 illustrates an exemplary system for assessing affective states of users based on data derived from, for example, wearable computing devices, according to some embodiments;

FIG. 3 illustrates another exemplary system for assessing affective states of users based on data derived from, for example, wearable computing devices, according to some embodiments;

FIG. 4 illustrates an exemplary affective state prediction unit for assessing affective states of a user in cooperation with a wearable computing device, according to some embodiments;

FIG. 5 illustrates sensors for use with an exemplary data-capable band as a wearable computing device;

FIG. 6 depicts a stressor analyzer configured to receive activity-related data to determine an affective state of a user, according to some embodiments;

FIGS. 7A and 7B depict examples of exemplary sensor data and relationships that can be used to determine an affective state of a user, according to some embodiments;

FIGS. 8A, 8B, and 8C depict applications generating data representing an affective state of a user, according to some embodiments;

FIG. 9 illustrates an exemplary affective state prediction unit disposed in a mobile computing device that operates in cooperation with a wearable computing device, according to some embodiments;

FIG. 10 illustrates an exemplary system for conveying affective states of a user to others, according to some embodiments;

FIG. 11 illustrates an exemplary system for detecting affective states of a user and modifying environmental characteristics in which a user is disposed responsive to the detected affective states of the user, according to some embodiments; and

FIG. 12 illustrates an exemplary computing platform to facilitate affective state assessments in accordance with various embodiments.

### DETAILED DESCRIPTION

Various embodiments or examples may be implemented in numerous ways, including as a system, a process, an apparatus, a user interface, or a series of program instructions on a computer readable medium such as a computer readable storage medium or a computer network where the program instructions are sent over optical, electronic, or wireless communication links. In general, operations of disclosed processes may be performed in an arbitrary order, unless otherwise provided in the claims.

A detailed description of one or more examples is provided below along with accompanying figures. The detailed description is provided in connection with such examples, but is not limited to any particular example. The scope is limited only by the claims and numerous alternatives, modifications, and equivalents are encompassed. Numerous specific details are set forth in the following description in order to provide a thorough understanding. These details are provided for the purpose of example and the described techniques may be practiced according to the claims without some or all of these specific details. For clarity, technical material that is known in the technical fields related to the examples has not been described in detail to avoid unnecessarily obscuring the description.

In some examples, the described techniques may be implemented as a computer program or application (hereafter “applications”) or as a plug-in, module, or sub-component of another application. The described techniques may be implemented as software, hardware, firmware, circuitry, or a combination thereof. If implemented as software, the described techniques may be implemented using various types of programming, development, scripting, or formatting languages, frameworks, syntax, applications, protocols, objects, or techniques, including ASP, ASP.net, .Net framework, Ruby, Ruby on Rails, C, Objective C, C++, C#, Adobe® Integrated Runtime™ (Adobe® AIR™), ActionScript™, Flex™, Lingo™, Java™, Javascript™, Ajax, Pert, COBOL, Fortran, ADA, XML, MXML, HTML, DHTML, XHTML, HTTP, XMPP, PHP, and others. The described techniques may be varied and are not limited to the embodiments, examples or descriptions provided.

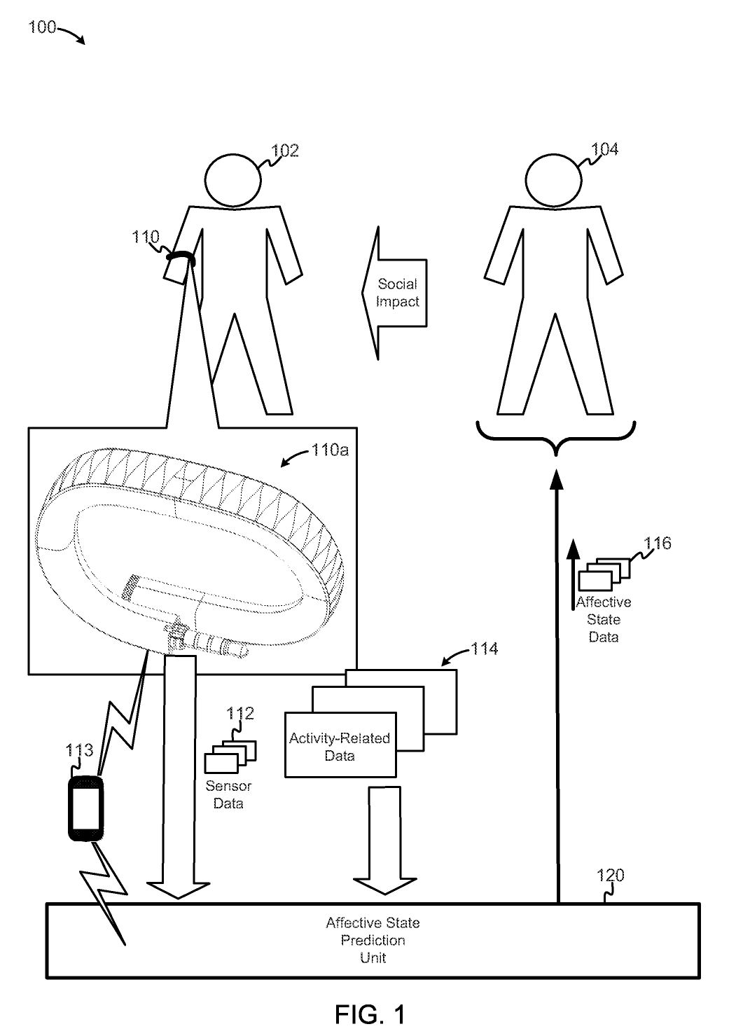

FIG. 1 illustrates an exemplary system for assessing affective states of a user based on data derived from, for example, a wearable computing device, according to some embodiments. Diagram 100 depicts a user 102 including a wearable device 110 interacting with a person 104. The interaction can be either bi-directional or unidirectional. As shown, at least person 104 is socially impacting user 102 or has some influence, by action or speech, upon the state of mind of user 102 (e.g., emotional state of mind). In some embodiments, wearable device 110 is a wearable computing device 110a that includes one or more sensors to detect attributes of the user, the environment, and other aspects of the interaction. Note that while FIG. 1 describes physiological changes, which can be detected, for user 102 responsive to person 104, the various embodiments are not limited as such and physiological states and conditions of user 102 can be determined regardless of the stimuli, which can include person 104 and other social factors (e.g., the social impact of one or more other people upon user 102, such as the type of people, friends, colleagues, audience members, etc.), environmental factor (e.g., the impact of one or more perceptible conditions of the environment in which user 102 is in, such as heat, humidity, sounds, etc.), situational factors (e.g., a situation under which user 102 can be subject to a stressor, such as trying to catch an airline flight, interviewing for a job, speaking in front of a crowd, being interrogated during a truth-determining proceeding, etc.), as well as any other factors.

Diagram 100 also depicts an affective state prediction unit 120 configured to receive sensor data 112 and activity-related data 114, and further configured to generate affective state data 116 to person 104 as emotive feedback describing the social impact of person 104 upon user 102. Affective state data 116 can be conveyed in near real-time or real time. Sensor data 112 includes data representing physiological information, such as skin conductivity, heart rate (“HR”), blood pressure (“BP”), heart rate variability (“HRV”), respiration rates, Mayer waves, which correlate with HRV, at least in some cases, body temperature, and the like. Further, sensor data 112 also can include data representing location (e.g., GPS coordinates) of user 102, as well as other environmental attributes in which user 102 is disposed that can affect the emotional state of user 102. Environmental attribute examples also include levels of background noise (e.g., loud, non-pleasurable noises can raise heart rates and stress levels), levels of ambient tight, number of people (e.g., whether the user is in a crowd), location of a user (e.g., at a dentist office, which tends to increase stress, at the beach, which tends to decrease stress, etc.), and other environmental factors, in some implementations, sensor data also can include motion-related data indicating accelerations and orientations of user 102 as determined by, for example, one or more accelerometers. Activity-related data 114 includes data representing primary activities (e.g., specific activities in which a user engages as exercise), sleep activities, nutritional activities, sedentary activities and other activities in which user 102 engages. Activity-related data 114 can represent activities performed during the interaction from person 104 to user 102, or at any other time period. Affective state prediction unit 120 uses sensor data 112 and activity-related data 114 to form affective state data 116. As used herein, the term “affective state” can refer, at least in some embodiments, to a feeling, a mood, and/or an emotional state of a user. In some cases, affective state data 116 includes data that predicts an emotion of user 102 or an estimated or approximated emotion or feeling of user 102 concurrent with and/or in response to the interaction with person 104 (or in response to any other stimuli). Affective state prediction unit 120 can be configured to generate data representing modifications in the affective state of user 102 responsive to changes in the interaction caused by person 104. As such, affective state data 116 provides feedback to person 104 to ensure that they are optimally interacting with user 102. In some embodiments, sensor data 112 can be communicated via a mobile communication and computing device 113. Further, affective state prediction unit 120 can be disposed in a mobile communication and computing device 113 or any other computing device. Further, the structures and/or functionalities of mobile communication and computing device 113 can be distributed among other computing devices over multiple devices (e.g., networked devices), according to some embodiments.

In some embodiments, affective state prediction unit 120 can be configured to use sensor data 112 from one or more sensors to determine an intensity of an affective state of user 102, and further configured to use activity-related data 114 to determine the polarity of the intensity of an affective state of user 102 (i.e., whether the polarity of the affective state is positive or negative). A low intensity (e.g., a calm state) of an affective state can coincide with less adrenaline and a low blood flow to the skin of user 102, whereas a high intensity (e.g., an aroused or stressed state) can coincide with high levels of adrenaline and a high blood flow to the skin (e.g., including an increase in perspiration). A high intensity can also be accompanied by increases in heart rate, blood pressure, rate of breathing, and the like, any of which can also be represented by or included in sensor data 112. A value of intensity can be used to determine an affective state or emotion, generally, too.

An affective state prediction unit 120 can be configured to generate affective state data 116 representing including a polarity of an affective state or emotion, such as either a positive or negative affective state or emotion. A positive affective state (“a good mood”) is an emotion or feeling that is generally determined to include positive states of mind (usually accompanying positive physiological attributes), such as happiness, joyfulness, being excited, alertness, attentiveness, among others, whereas a negative affective state (“a bad mood”) is an emotion or feeling that is generally determined to include negative states of mind (usually accompanying negative physiological attributes), such as anger, agitation, distress, disgust, sadness, depression, among others. Examples of positive affective states having high intensities can include happiness and joyfulness, whereas an example of low positive affective states includes states of deep relaxation. Examples of negative affective states having high intensities can include anger and distress, whereas an example of low negative affective states includes states of depression. According to some embodiments, affective state prediction unit 120 can predict an emotion at a finer level of granularity of the positive or negative affective state. For example, affective state prediction unit 120 can approximate a user's affective state as one of the four following: a high-intensive negative affective state, a low-intensive negative affective state, a low-intensive positive affective state, and a high-intensive positive affective state. In other examples, affective state prediction unit 120 can approximate a user's emotion, such as happiness, anger, sadness, etc.

Wearable device 110a is configured to dispose sensors (e.g., physiological sensors) at or adjacent distal portions of an appendage or limb. Examples of distal portions of appendages or limbs include wrists, ankles, toes, fingers, and the like. Distal portions or locations are those that are furthest away from, for example, a torso relative to the proximal portions or locations. Proximal portions or locations are located at or near the point of attachment of the appendage or limb to the torso or body. In some cases, disposing the sensors at the distal portions of a limb can provide for enhanced sensing as the extremities of a person's body may exhibit the presence of an infirmity, ailment or condition more readily than a person's core (i.e., torso).

In some embodiments, wearable device 110a includes circuitry and electrodes (not shown) configured to determine the bioelectric impedance (“bioimpedance”) of one or more types of tissues of a wearer to identify, measure, and monitor physiological characteristics. For example, a drive signal having a known amplitude and frequency can be applied to a user, from which a sink signal is received as bioimpedance signal. The bioimpedance signal is a measured signal that includes real and complex components. Examples of real components include extra-cellular and intra-cellular spaces of tissue, among other things, and examples of complex components include cellular membrane capacitance, among other things. Further, the measured bioimpedance signal can include real and/or complex components associated with arterial structures (e.g., arterial cells, etc.) and the presence (or absence) of blood pulsing through an arterial structure. In some examples, a heart rate signal, or other physiological signals, can be determined (i.e., recovered) from the measured bioimpedance signal by, for example, comparing the measured bioimpedance signal against the waveform of the drive signal to determine a phase delay (or shift) of the measured complex components. The bioimpedance sensor signals can provide a heart rate, a respiration rate, and a Mayer wave rate.

In some embodiments, wearable device 110a can include a microphone (not shown) configured to contact (or to be positioned adjacent to) the skin of the wearer, whereby the microphone is adapted to receive sound and acoustic energy generated by the wearer (e.g., the source of sounds associated with physiological information). The microphone can also be disposed in wearable device 110a. According to some embodiments, the microphone can be implemented as a skin surface microphone (“SSM”), or a portion thereof, according to some embodiments. An SSM can be an acoustic microphone configured to enable it to respond to acoustic energy originating from human tissue rather than airborne acoustic sources. As such, an SSM facilitates relatively accurate detection of physiological signals through a medium for which the SSM can be adapted (e.g., relative to the acoustic impedance of human tissue). Examples of SSM structures in which piezoelectric sensors can be implemented (e.g., rather than a diaphragm) are described in U.S. patent application Ser. No. 11/199,856, filed on Aug. 8, 2005, and U.S. patent application Ser. No. 13/672,398, filed on Nov. 8, 2012, both of which are incorporated by reference. As used herein, the term human tissue can refer to, at least in some examples, as skin, muscle, blood, or other tissue. In some embodiments, a piezoelectric sensor can constitute an SSM. Data representing one or more sensor signals can include acoustic signal information received from an SSM or other microphone, according to some examples.

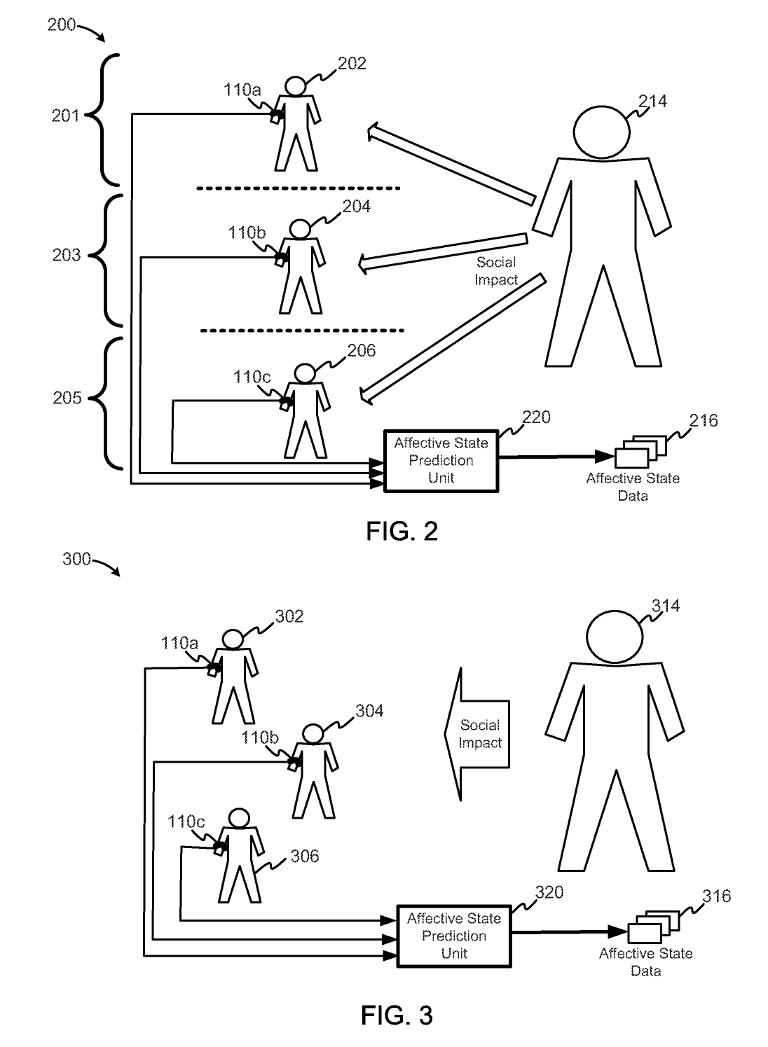

FIG. 2 illustrates an exemplary system for assessing affective states of users based on data derived from, for example, wearable computing devices, according to some embodiments. Diagram 200 depicts users 202, 204, and 206 including wearable devices 110a, 110b, and 110c, respectively, whereby each of the users interact with a person 214 at different time intervals. For example, person 214 interacts with user 202 during time interval 201, with user 204 during time interval 203, and with user 206 during time interval 205. Data retrieved from wearable devices 110a, 110b, and 110c can be used by affective state prediction unit 220 to generate affective state data 216. Person 214 can consume affective state data 216 as feedback to improve or enhance the social interaction of person 204 with any of users 202, 204, and 206. For example, the system depicted in diagram 200 can be used to coach or improve executive or enterprise interpersonal interactions.

FIG. 3 illustrates another exemplary system for assessing affective states of users based on data derived from, for example, wearable computing devices, according to some embodiments. Diagram 300 depicts users 302, 304, and 306 including wearable devices 110a, 110b, and 110c, respectively, whereby the users interact with a person 314 concurrently (or nearly so). For example, person 314 interacts with user 302, user 304, and user 306 during, for example, a presentation by person 314 to an audience including user 302, user 304, and user 306. Data retrieved from wearable devices 110a, 110b, and 110c can be used by affective state prediction unit 320 to generate affective state data 316, which can represent data for either individuals or the audience collectively. For example, affective state data 316 can represent an aggregated emotive score that represents a collective feeling or mood toward either the information being presented or in the manner in which it is presented. Person 314 can consume affective state data 316 as feedback to improve or enhance the social interaction between person 304 and any of users 302, 304, and 306 (e.g., to make changes in the presentation in real-time or for future presentations).

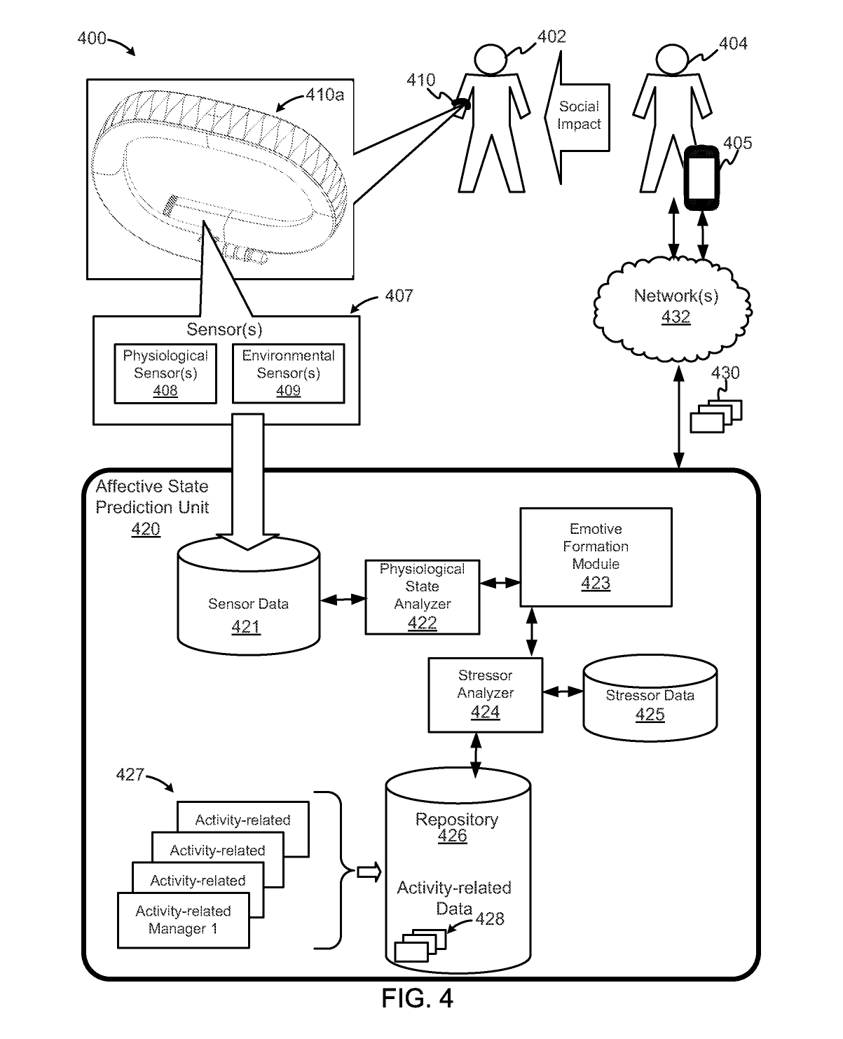

FIG. 4 illustrates an exemplary affective state prediction unit for assessing affective states of a user in cooperation with a wearable computing device, according to some embodiments. Diagram 400 depicts a user 402 including a wearable device 410 interacting with a person 404. The interaction can be either bi-directional or unidirectional. In some cases, the degree to which person 404 is socially impacting user 402, as well as the quality of the interaction, is determined by affective state prediction unit 420. In some embodiments, wearable device 410 is a wearable computing device 410a that includes one or more sensors to detect attributes of the user, the environment, and other aspects of the interaction. As shown, wearable computing device 410a includes one or more sensors 407 that can include physiological sensor(s) 408 and environmental sensor(s) 409.

According to some embodiments, affective state prediction unit 420 includes a repository 421 including sensor data from, for example, wearable device 410a or any other device. Also included is a physiological state analyzer 422 that is configured to receive and analyze the sensor data to compute a sensor-derived value representative of an intensity of an affective state of user 402. In some embodiments, the sensor-derived value can represent an aggregated value of sensor data (e.g., an aggregated value of sensor data value). Affective state prediction unit 420 can also include a number of activity-related managers 427 configured to generate activity-related data 428 stored in a repository 426, which, in turn, is coupled to a stressor analyzer 424. Stressor analyzer 424 is coupled to a repository 425 for storing stressor data.

One or more activity-related managers 427 are configured to receive data representing parameters relating to one or more motion or movement-related activities of a user and to maintain data representing one or more activity profiles. Activity-related parameters describe characteristics, factors or attributes of motion or movements in which a user is engaged, and can be established from sensor data or derived based on computations. Examples of parameters include motion actions, such as a step, stride, swim stroke, rowing stroke, bike pedal stroke, and the like, depending on the activity in which a user is participating. As used herein, a motion action is a unit of motion (e.g., a substantially repetitive motion) indicative of either a single activity or a subset of activities and can be detected, for example, with one or more accelerometers and/or logic configured to determine an activity composed of specific motion actions. According to some examples, activity-related managers 427 can include a nutrition manager, a sleep manager, an activity manager, a sedentary activity manager, and the like, examples of which can be found in U.S. patent application Ser. No. 13/433,204, filed on Mar. 28, 2012 having Attorney Docket No. ALI-013CIP1; U.S. patent application Ser. No. 13/433,208, filed Mar. 28, 2012 having Attorney Docket No. ALI-013CIP2; U.S. patent application Ser. No. 13/433,208, filed Mar. 28, 2012 having Attorney Docket No. ALI-013CIP3; U.S. patent application Ser. No. 13/454,040, filed Apr. 23, 2012 having Attorney Docket No. ALI-013CIP1CIP1; and U.S. patent application Ser. No. 13/627,997, filed Sep. 26, 2012 having Attorney Docket No. ALI-100, all of which are incorporated herein by reference.

In some embodiments, stressor analyzer 424 is configured to receive activity-related data 428 to determine stress scores that weigh against a positive affective state in favor of a negative affective state. For example, if activity-related data 428 indicates user 402 has had little sleep, is hungry, and has just traveled a great distance, then user 402 is predisposed to being irritable or in a negative frame of mine (and thus in a relatively “bad” mood). Also, user 402 may be predisposed to react negatively to stimuli, especially unwanted or undesired stimuli that can be perceived as stress. Therefore, such activity-related data 428 can be used to determine whether an intensity derived from physiological state analyzer 422 is either negative or positive.

Emotive formation module 433 is configured to receive data from physiological state analyzer 422 and/or stressor analyzer 424 to predict an emotion in which user 402 is experiencing (e.g., as a positive or negative affective state). Affective state prediction unit 420 can transmit affective state data 430 via network(s) 432 to person 404 (or a computing device thereof) as emotive feedback. Note that in some embodiments, physiological state analyzer 422 is sufficient to determine affective state data 430. For example, a bio-impedance received sensor signal can be sufficient to extract heart-related physiological signals that can be used to determine intensities as well as positive or negative intensities. For example, HRV (e.g., based on Mayer waves) can be used to determine positive or negative intensities associated with positive or negative affective states. in other embodiments, stressor analyzer 424 is sufficient to determine affective state data 430. In various embodiments, physiological state analyzer 422 and stressor analyzer 424 can be used in combination or with other data or functionalities to determine affective state data 430. In some embodiments, affective state data 430 is configured to establish communications with wearable device 410a for receiving affective state data into a computing device 405, which is associated with (and accessible by) person 404. In response, person 404 can modify his or her social interactions with user 402 to improve the affective state of user 402. Computing device 405 can be a mobile phone or computing device, or can be another wearable device 410a.

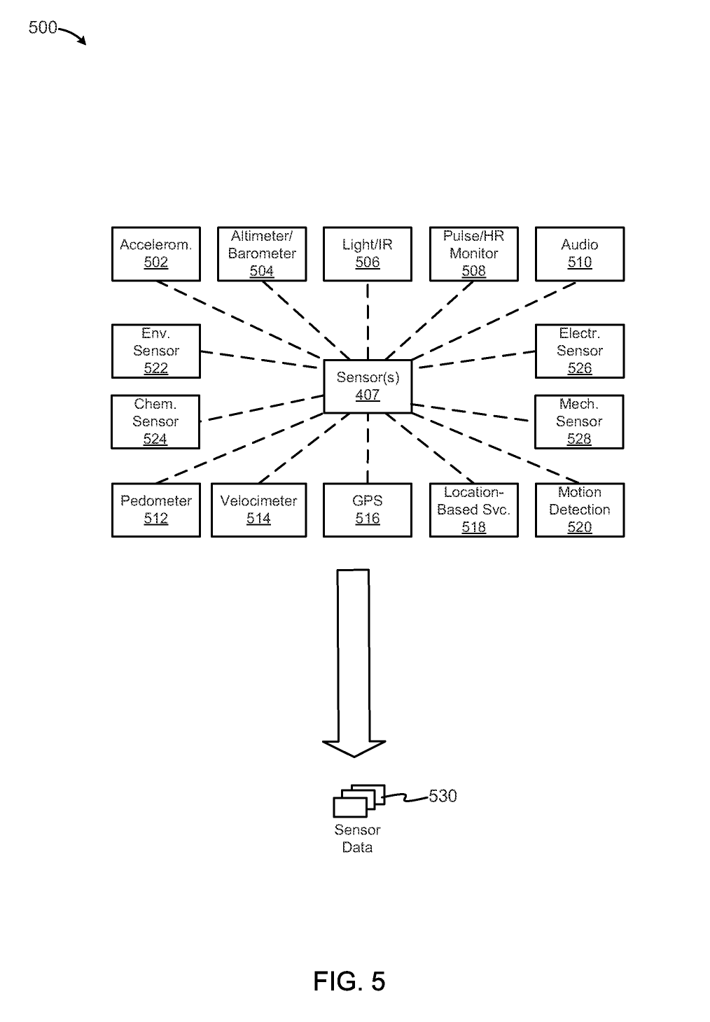

FIG. 5 illustrates sensors for use with an exemplary data-capable band as a wearable computing device. Sensor 407 can be implemented using various types of sensors, some of which are shown, to generate sensor data 530 based on one or more sensors. Like-numbered and named elements can describe the same or substantially similar element as those shown in other descriptions. Here, sensor(s) 407 can be implemented as accelerometer 502, altimeter/barometer 504, light/infrared (“IR”) sensor 506, pulse/heart rate (“HR”) monitor 508, audio sensor 510 (e.g., microphone, transducer, or others), pedometer 512, velocimeter 514, GPS receiver 516, location-based service sensor 518 (e.g., sensor for determining location within a cellular or micro-cellular network, which may or may not use GPS or other satellite constellations for fixing a position), motion detection sensor 520, environmental sensor 522, chemical sensor 524, electrical sensor 526, or mechanical sensor 528.

As shown, accelerometer 502 can be used to capture data associated with motion detection along 1, 2, or 3-axes of measurement, without limitation to any specific type of specification of sensor. Accelerometer 502 can also be implemented to measure various types of user motion and can be configured based on the type of sensor, firmware, software, hardware, or circuitry used. As another example, altimeter/barometer 504 can be used to measure environment pressure, atmospheric or otherwise, and is not limited to any specification or type of pressure-reading device. In some examples, altimeter/barometer 504 can be an altimeter, a barometer, or a combination thereof. For example, altimeter/barometer 504 can be implemented as an altimeter for measuring above ground level (“AGL”) pressure in a wearable computing device, which has been configured for use by naval or military aviators. As another example, altimeter/barometer 504 can be implemented as a barometer for reading atmospheric pressure for marine-based applications. In other examples, altimeter/barometer 504 can be implemented differently.

Other types of sensors that can be used to measure light or photonic conditions include light/IR sensor 506, motion detection sensor 520, and environmental sensor 522, the latter of which can include any type of sensor for capturing data associated with environmental conditions beyond light. Further, motion detection sensor 520 can be configured to detect motion using a variety of techniques and technologies, including, but not limited to comparative or differential light analysis comparing foreground and background lighting), sound monitoring, or others. Audio sensor 510 can be implemented using any type of device configured to record or capture sound.

In some examples, pedometer 512 can be implemented using devices to measure various types of data associated with pedestrian-oriented activities such as running or walking. Footstrikes, stride length, stride length or interval, time, and other motion action-based data can be measured. Velocimeter 514 can be implemented, in some examples, to measure velocity speed and directional vectors) without limitation to any particular activity. Further, additional sensors that can be used as sensor 407 include those configured to identify or obtain location-based data. For example, GPS receiver 516 can be used to obtain coordinates of the geographic location of a wearable device using, for example, various types of signals transmitted by civilian and/or military satellite constellations in low, medium, or high earth orbit (e.g., “LEO,” “MEO,” or “GEO”). In other examples, differential GPS algorithms can also be implemented with GPS receiver 516, which can be used to generate more precise or accurate coordinates. Still further, location-based services sensor 518 can be implemented to obtain location-based data including, but not limited to location, nearby services or items of interest, and the like. As an example, location-based services sensor 518 can be configured to detect an electronic signal, encoded or otherwise, that provides information regarding a physical locale as band 200 passes. The electronic signal can include, in some examples, encoded data regarding the location and information associated therewith. Electrical sensor 526 and mechanical sensor 528 can be configured to include other types (e.g., haptic, kinetic, piezoelectric, piezomechanical, pressure, touch, thermal, and others) of sensors for data input to a wearable device, without limitation. Other types of sensors apart from those shown can also be used, including magnetic flux sensors such as solid-state compasses and the like, including gyroscopic sensors. While the present illustration provides numerous examples of types of sensors that can be used with a wearable device, others not shown or described can be implemented with or as a substitute for any sensor shown or described.

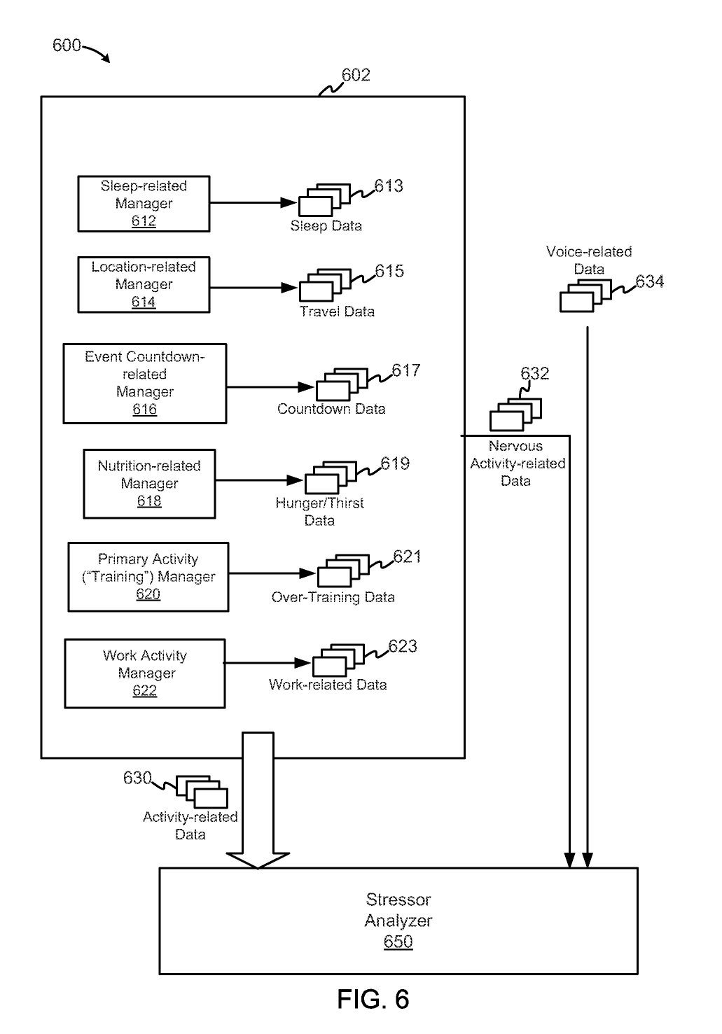

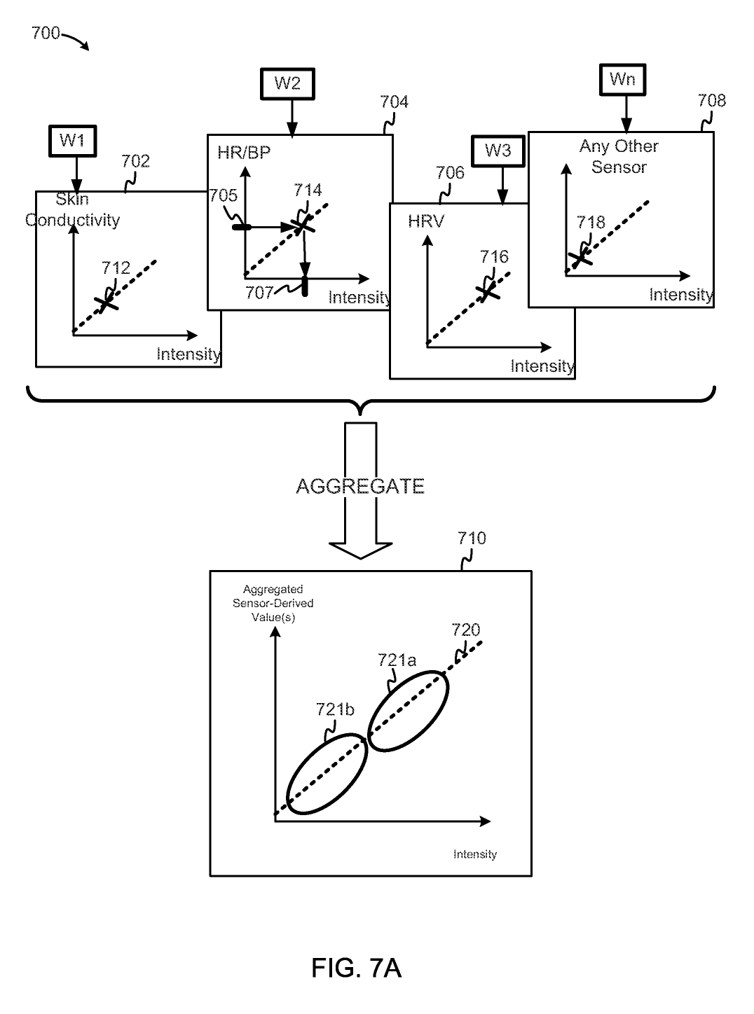

FIG. 6 depicts a stressor analyzer configured to receive activity-related data to determine an affective state of a user, according to some embodiments. Activity-related managers 602 can include any number of activity-related managers. Sleep-related manager 612 is configured to generate sleep data 613 indicating various gradations of sleep quality for a user. For example, sleep scores indicating the user is well-rested are likely to urge a user toward a positive affective state, whereas poor sleep scores likely predisposes the user to irritability and negative affective states (e.g., in which users are less tolerable to undesired stimuli). Location-related manager 614 is configured to generate travel data 615 indicating various gradations of travel by a user (e.g., from heavy and long travel to light and short travel). For example, travel scores indicating the user has traveled 10 hours in an airplane flight, which is likely predisposed to make a user irritable, likely will have values that likely describes a user as being associated with a negative state. Event countdown-related manager 616 is configured to generate countdown data 617 indicating an amount of time before the user participates in an event. As the time decreases to an event, a user is more likely to be exposed to situational stress, such as when a user is trying to catch an airplane flight and time is growing short. Such stress is low 24 hours before, but increases to two hours before the flight when the user is perhaps stuck in traffic on the way to the airport. Nutrition-related manager 618 is configured to generate hunger/thirst data 619 indicating various gradations of nutrition quality for a user. For example, nutrition scores indicating the user is well-nourished are likely to urge a user toward a positive affective state, whereas poor nutrition scores (i.e., poor nourishment) likely predisposes the user to acrimony and negative affective states. Primary manager 620 is configured to generate over-training data 621 indicating various gradations of over-training for a user. For example, over-training scores indicating the user has stressed the body as a result of over-training likely predisposes the user to duress, distress, or negative affective states. Work activity manager 622 is configured to generate work-related data 623 indicating various gradations of hours worked by a user. For example, a user may be under a lot of stress after working long, hard hours, which, in turn, likely predisposes the user to duress or negative affective states. Other types of activities and activity-related data can be generated by activity-related managers 602 and are not limited to those described herein.

Stressor analyzer 650 is configured to receive the above-described data as activity-related data 630 for generating a score that indicates likely positive or negative affective states of a user. In some embodiments, nervous activity-related data 632 can be received. This data describes one or more nervous motions (e.g., fidgeting) that can indicate that the user is likely experiencing negative emotions. Voice-related data 634 is data gathered from audio sensors or in a mobile phone, or by other means. Voice-related data 634 can represent data including vocabulary that is indicative of a state of mind, as well as the tone, pitch, volume and speed of the user's voice. Stressor analyzer 650, therefore, can generate data representing the user's negative or positive state of emotion.

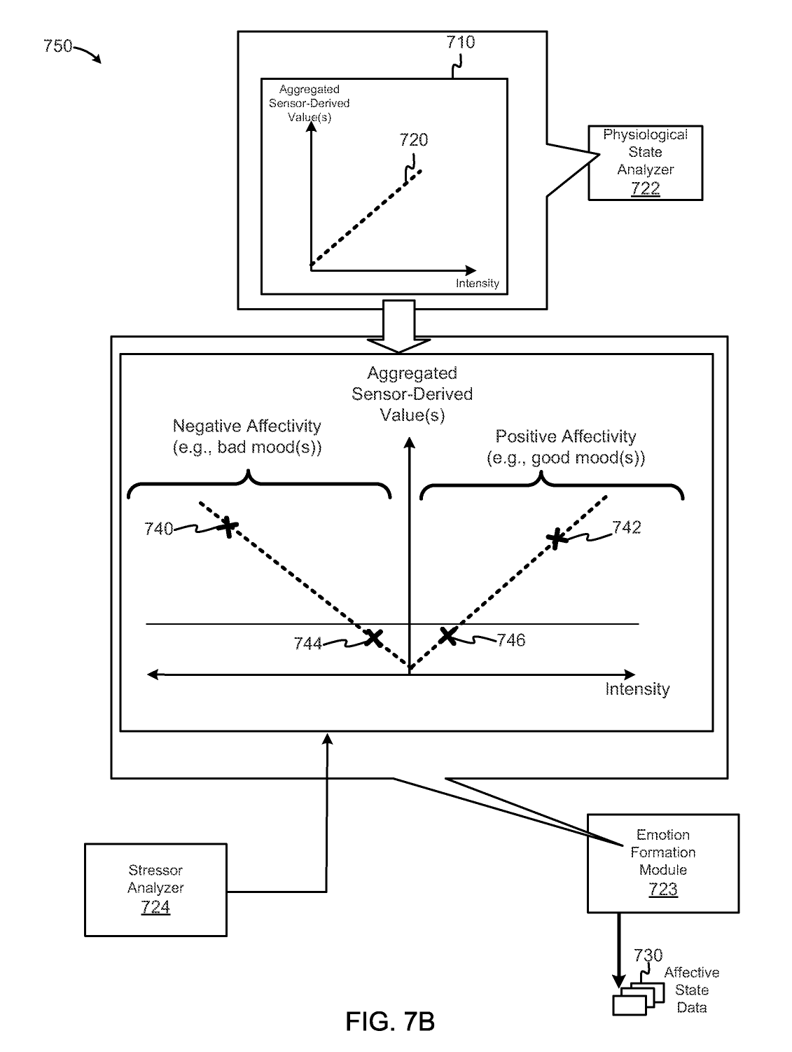

FIGS. 7A and 7B depict examples of exemplary sensor data and relationships that can be used to determine an affective state of a user, according to some embodiments. Diagram 700 of FIG. 7A depicts a number of sensor relationships 702 to 708 that can generate sensor data, according to some embodiments. Note that sensor relationships 702 to 708 are shown as linear for ease of discussion, but need not be so limited (i.e., one or more of sensor relationships 702 to 708 can be non-linear). For example, a galvanic skin response (“GSR”) sensor can provide for sensor data 702 (e.g., instantaneous or over specific durations of time of any length), a heart rate (“HR”) sensor can provide for sensor data 704, a heart rate variability (“HRV”) sensor can provide for sensor data 706 depicting variability in heart rate. In the example shown, relative values of the physical characteristics can be associated with sensor data 702, 704, 706, and 708, and can be depicted as values 712, 714, 716, and 718. To determine the contribution of heart rate (“HR”), a sensed heart rate 705 applied to sensor relationship 704 provides for an intensity value 707, which can be a contribution (weighted or unweighted) to the determination of the aggregated intensity based on the combination of intensities determined by sensor relationships 702 to 708. In some cases, these values can be normalized to be additive or weighted by a weight factor, such as weighting factors W1, W2, W3, and Wn. Therefore, in some cases, weighted values of 712, 714, 716, and 718 can be used (e.g., added) to form an aggregated sensor-derived value that can be plotted as aggregated sensor-derived value 720. Region 721b indicates a relatively low-level intensity of the aggregated sensor-derived value, whereas region 711a indicates a relatively high-level intensity.

Note that in some cases, lower variability in heart rate can indicate negative affective states, whereas higher variability in heart rate can indicate positive affective states. In some examples, the term “heart rate variability” can describe the variation of a time interval between heartbeats. HRV can describe a variation in the beat-to-beat interval and can be expressed in terms of frequency components (e.g., low frequency and high frequency components), at least in some cases. In some examples, Mayer waves can be detected as sensor data 702, which can be used to determine heart rate variability (“HRV”), as heart rate variability can be correlated to Mayer waves. Further, affective state prediction units, as described herein, can use, at least in some embodiments, HRV to determine an affective state or emotional state of a user. Thus, HRV may be used to correlate with an emotion state of the user.

Other sensors can provide other sensor data 708. An aggregated sensor-derived value having relationship 720 is computed as an aggregated sensor 710. Note that in various embodiments one or more subsets of data from one or more sensors can be used, and thus are not limited to aggregation of data from different sensors. As shown in FIG. 7B, aggregated sensor-derived value 720 can be generated by a physiological state analyzer 722 indicating a level of intensity. Stressor analyzer 724 is configured to determine whether the level of intensity is within a range of negative affectivity or is within a range of positive affectivity. For example, an intensity 740 in a range of negative affectivity can represent an emotional state similar to, or approximating, distress, whereas intensity 742 in a range of positive affectivity can represent an emotional state similar to, or approximating, happiness. As another example, an intensity 744 in a range of negative affectivity can represent an emotional state similar to, or approximating, depression/sadness, whereas intensity 746 in a range of positive affectivity can represent an emotional state similar to, or approximating, relaxation. As shown, intensities 740 and 742 are greater than that of intensities 744 and 746. Emotive formulation module 723 is configured to transmit this information as affective state data 730 describing a predicted emotion of a user.

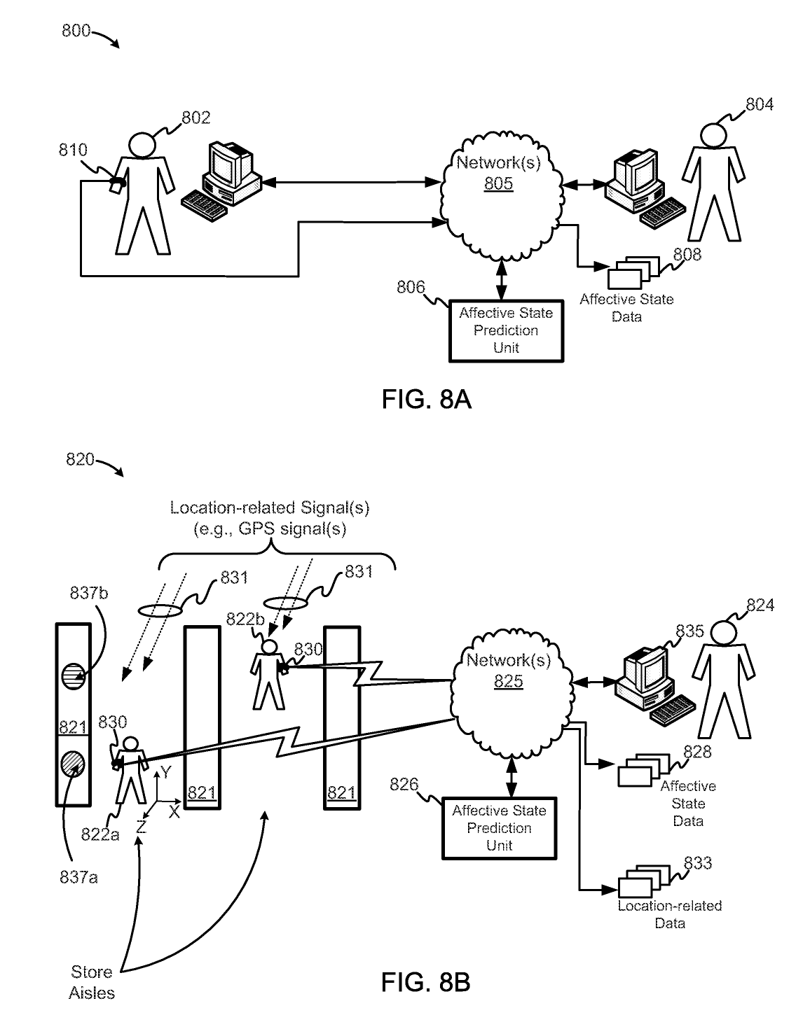

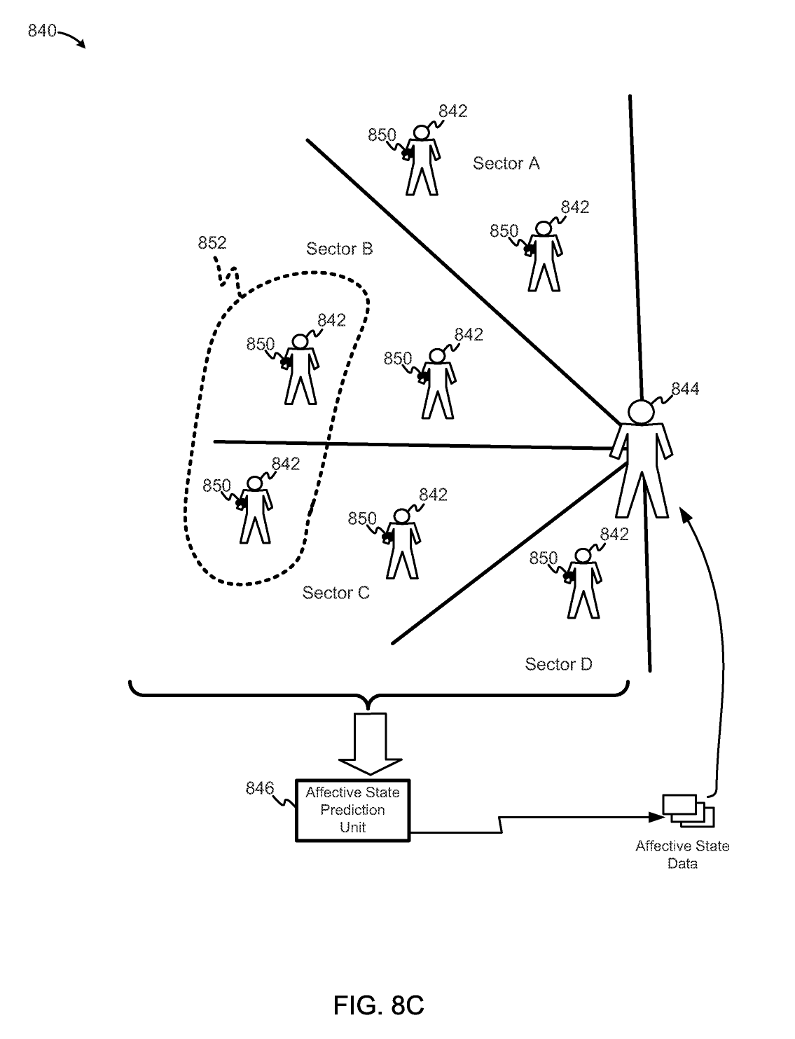

FIGS. 8A, 8B, and 8C depict applications generating data representing an affective state of a user, according to some embodiments. Diagram 800 of FIG. 8A depicts a person 804 interacting via a networks 805 with a user 802 including a wearable device 810, according to some embodiments. Affective state data associated with user 802 was generated by affective state prediction unit 806 to send affective state data 808 to person 804. In this example, person 804 can be a customer service representative interacting with user 802 as a customer. The experience (either positive or negative) can be fed back to the customer service representative to ensure the customer's needs are met.

Diagram 820 of FIG. 8B depicts a person 824 monitoring via a networks 825 affective states of a number of users 822 each including a wearable device 830, according to some embodiments. In this example, users 822 (e.g., users 822a and 822b) can be in various aisles of a store (e.g., retail store, grocery store, etc.). For example, any of users 822 emoting frustration or anger can be sensed by affective state prediction unit 826, which forwards this data as affective state data 828 to person 824. In this example, person 824 can assist user 822 to find the products or items (e.g., groceries) they are seeking at locations in shelves 821. Wearable device 830 can be configured to determine a location of a user 830 using any of various techniques of determining the location, such as dead reckoning or other techniques. According to various embodiments, wearable devices 830 can be configured to receive location-related signals 831, such as Global Positioning System (“GPS”) signals, to determine an approximate location of users 822 relative to items in a surrounding environment. For example, affective state prediction unit 826 can be configured also to transmit location-related data 833 (e.g., GPS coordinates or the like) associated with affective state data 828 to a computing device 835, which can be associated with person 824. Therefore, affective state prediction unit 826 can be configured to determine a reaction (e.g., an emotive reaction) of user 822a to an item, such as a product, placed at position 837. Such a reaction can be indicated by affective state data 828, which can be used (e.g., over a number of samples of different users 822) to gather information to support decisions of optimal product placement (e.g., general negative reactions can prompt person 824 or an associated entity to remove an item of lower average interest, such as an item disposed at location 837b, and replace it with items having the capacity to generate more positive reactions). Purchasing data (not shown), such as data generated at a check-out register or a scanner), can be used to confirm affective state data 828 for a specific item location associated with the purchased item rather than other item locations having items that were not purchased). According to at least some embodiments, wearable device 830 can include orientation-related sensors (e.g., gyroscopic sensors or any other devices and/or logic for determining orientation of user 822) to assist in determining a direction in which user 822a, for example, is viewing. By using the aforementioned devices and techniques, person 824 or an associated entity can make more optimal product placement decisions as well as customer assistance-related actions.

Diagram 840 of FIG. 8C depicts a person 844 monitoring a number of users 842 including a wearable device 850, according to some embodiments. In this example, users 842 are in different sectors of an audience listening to a presentation. Different groups of users 842 can emote differently. For instance, users 842 in portion 852 may emote distress if, for example, they are having difficulty hearing. In this case, affective state prediction unit 846 can provide affective state data of users 842 in portion 852 to person 844 so that the presentation can be modified (e.g., increased volume or attention) to accommodate those users 842.

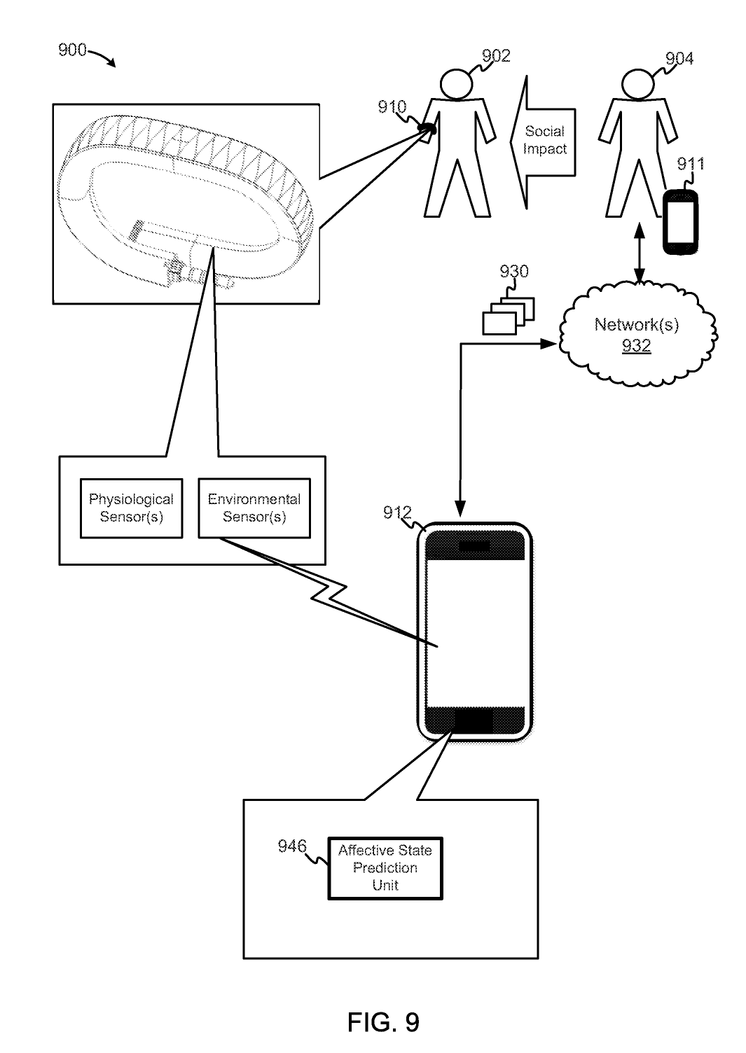

FIG. 9 illustrates an exemplary affective state prediction unit disposed in a mobile computing device that operates in cooperation with a wearable computing device, according to some embodiments. Diagram 900 depicts a user 902 including a wearable device 910 interacting with a person 904. In some cases, the degree to which person 904 is socially impacting user 902 of interest is identified by affective state prediction unit 946, which is disposed in mobile device 912, such as a mobile smart phone. Note that in some embodiments, affective state prediction unit 946 can be disposed as computing device 911, which is associated with and accessible by person 904.

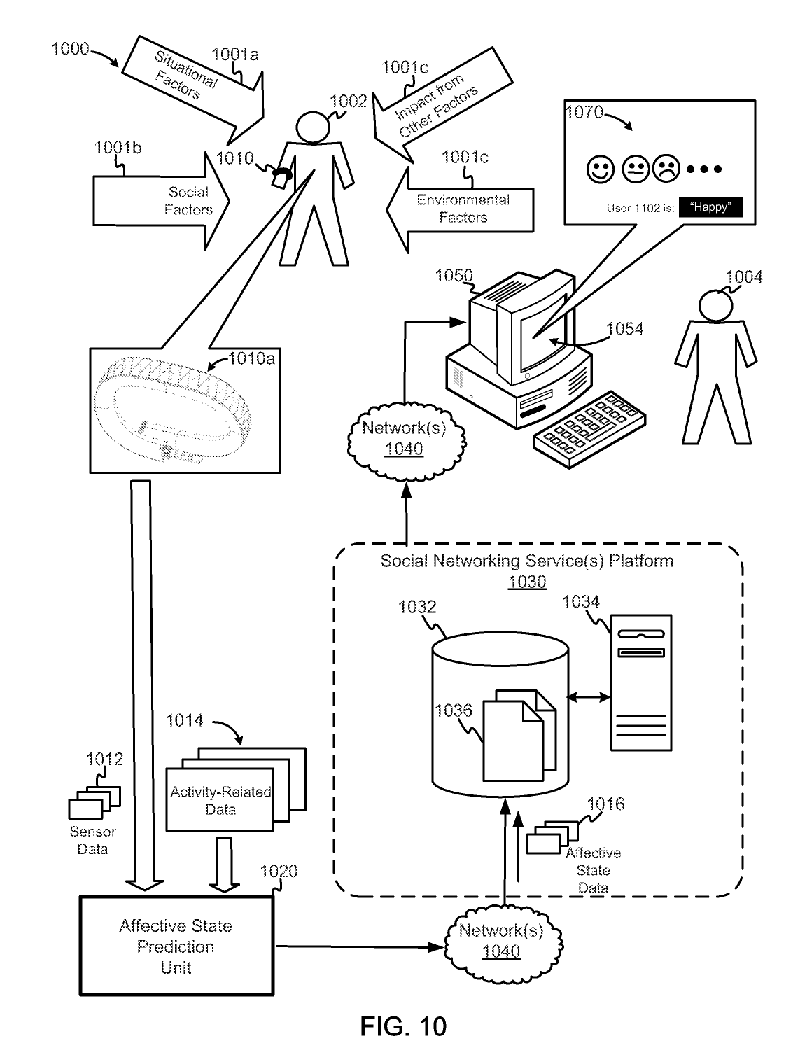

FIG. 10 illustrates an exemplary system for conveying affective states of a user to others, according to some embodiments. The affective states of the user can be based on data derived from, for example, a wearable computing device 1010. Diagram 1000 depicts a user 1002 being subject to various external and/or internals conditions in which user 1002 reacts physiologically in a manner that can be consistent with one or more emotions and/or moods. For example, user 1002 can be subject to various factors that can influence an emotion or mood of user 1002, including situational factors 1001a (e.g., a situation under which user 1002 can be subject to a stressor, such as trying to catch an airline flight), social factors 1001b (e.g., the social impact of one or more other people upon user 1002), environmental factors 1001c (e.g., the impact of one or more perceptible conditions of the environment in which user 1002 is in), and the impact of other factors 1001c. As described in FIG. 1, wearable device 1010 can be a wearable computing device 1010a that includes one or more sensors to detect attributes of the user, the environment, and other aspects of the interaction.

Similar to FIG. 1, at least in some respects, diagram 1000 also depicts an affective state prediction unit 1020 configured to receive sensor data 1012 and activity-related data 1014, and further configured to generate affective state data 1016. To convey the affective state of user 1002, affective state data 1016 can be communicated to person 1004 or, as shown, to a social networking service (“SNS”) platform 1030 via one or more networks 1040. Examples of SNS platform 1030 can include, for instance, Facebook®, Yahoo! IM™, GTalk™, MSN Messenger™, Twitter® and other private or public social networks. Social networking service platform 1030 can include a server 1034 including processors and/or logic to access data representing a file 1036 in a repository 1032. The data representing file 1036 includes data associated with user 1002, including socially-related data (e.g., friend subscriptions, categories of interest, etc.). The data representing file 1036 can also include data specifying authorization by person 104 (e.g., a friend) to access the social web page of user 1002, as generated by SNS platform 1030. In one example, affective state data 1016 is used to update the data representing file 1034 to indicate a detected mood or emotion of user 1002. The processors and/or logic in server 1034 can be configured to associate one or more symbols representing the detected mood or emotion of user 1002, and can be further configured to transmit data representing one or more symbols 1070 (e.g., graphical images, such as emoticons, text, or any other type of symbol) for presentation of the symbols, for instance, on a display 1054 of a computing device 1050. Therefore, a person 1004 can discern the mood and/or emotional state of user 1002, whereby person can reach out to user 1002 to assist or otherwise communicate with user 1002 based on the mood or emotional state of user 1002.

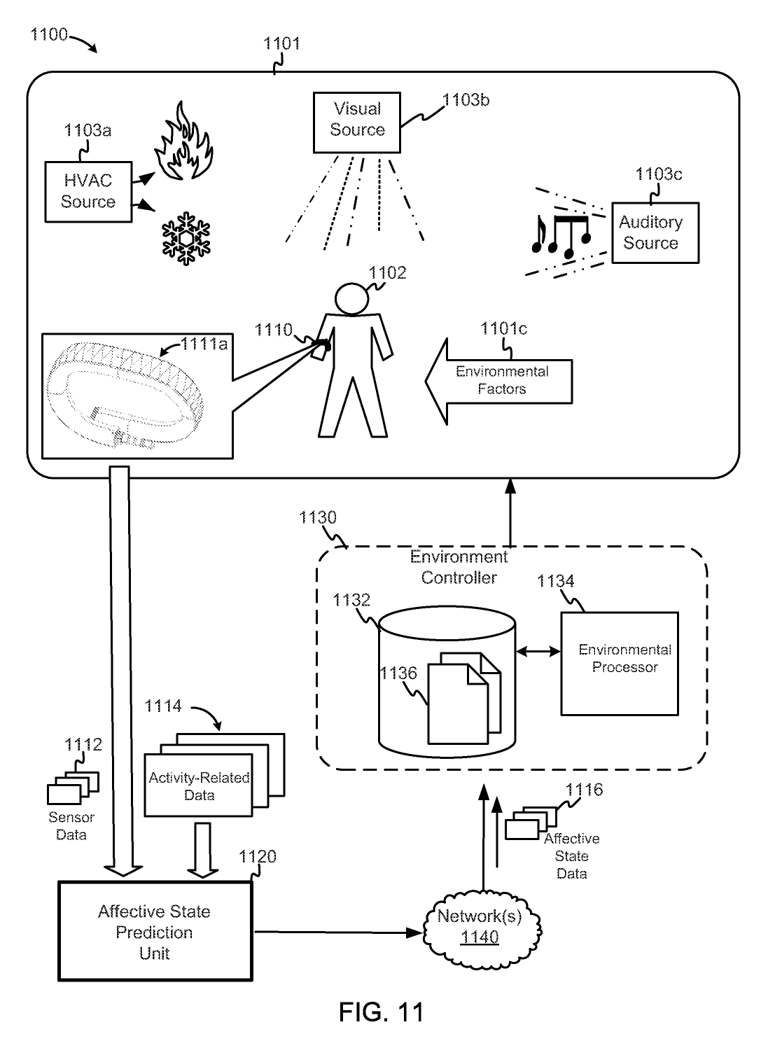

FIG. 11 illustrates an exemplary system for detecting affective states of a user and modifying environmental characteristics in which a user is disposed responsive to the detected affective states of the user, according to some embodiments. As with FIG. 10, the affective states of the user can be based on data derived from, for example, a wearable computing device 1110. Diagram 1100 depicts a user 1102 being subject to environmental factors 1101c in an environment 1101, including one or more perceptible conditions of the environment that can affect the mood or emotional state of user 1102. As described in FIG. 1, wearable device 1110 can be a wearable computing device 1110a that includes one or more sensors to detect attributes of the user, the environment, and other aspects of the interaction.

Similar to FIG. 1, at least in some respects, diagram 1100 also depicts an affective state prediction unit 1120 configured to receive sensor data 1112 and activity-related data 1114, and further configured to generate affective state data 1116. The affective state data 1116 can be transmitted via networks 1140 (or any other communication channel) to an environmental controller 1130, which includes an environment processor 1134 and a repository 1132 configured to store data files 1136. Environment processor 1134 is configured to analyze affective state data 1116 to determine an approximate mood or emotional state of user 1102, and is further configured to identify one or more data files 1136 associated with the approximate mood or emotional state, Data files 1136 can store data representing instructions for activating one or more sources that can modify one or more environmental factors 1101c in response to a determined mood and/or emotional state. Examples of sources that can influence environmental factors 1101c include an auditory source 1103c, such as a music-generating device (e.g., a digital receiver or music player), a visual source 1103b, such as variable lighting, imagery (e.g., digital pictures, motifs, or video), a heat, ventilation and air conditioning unit (“HVAC”) controller (e.g., a thermostat), or any other source. In operation, environmental controller 1130 can determine the mood or emotional state of user 1102 and adjust the surroundings of the user to, for example, cheer up the user 1102 if the user is depressed. If the user is tired and ought to get some sleep, the auditory source 1103c can play appropriate soundscape or relaxing music, the visual source 1103b can dim the lighting, and HVAC source 1103a can set the ambient temperature to one conducive to sleep. But if the user is excited and likely happy, the auditory source 1103c can play energetic music, the visual source 1103b can brighten the lighting, and HVAC source 1103a can set the ambient temperature to one conducive to staying awake and enjoying the mood.

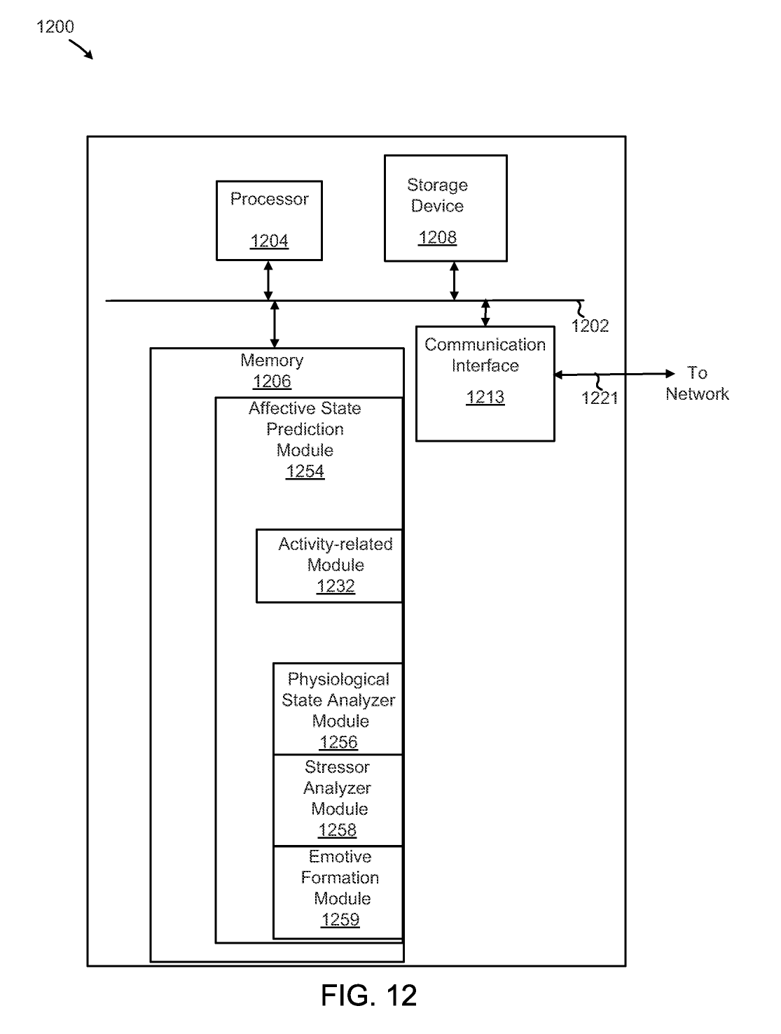

FIG. 12 illustrates an exemplary computing platform in accordance with various embodiments. In some examples, computing platform 1200 may be used to implement computer programs, applications, methods, processes, or other software to perform the above-described techniques. Computing platform 1200 includes a bus 1202 or other communication mechanism for communicating information, which interconnects subsystems and devices, such as processor 1204, system memory 1206 (e.g., RAM), storage device 1208 (e.g., ROM), a communication interface 1213 (e.g., an Ethernet or wireless controller) to facilitate communications via a port on communication link 1221 to communicate, for example, with a wearable device.

According to some examples, computing platform 1200 performs specific operations by processor 1204 executing one or more sequences of one or more instructions stored in system memory 1206. Such instructions or data may be read into system memory 1206 from another computer readable medium, such as storage device 1208. In some examples, hard-wired circuitry may be used in place of or in combination with software instructions for implementation. Instructions may be embedded in software or firmware. The term “computer readable medium” refers to any tangible medium that participates in providing instructions to processor 1204 for execution. Such a medium may take many forms, including but not limited to, non-volatile media and volatile media. Non-volatile media includes, for example, optical or magnetic disks and the like. Volatile media includes dynamic memory, such as system memory 1206.

Common forms of computer readable media includes, for example, floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, or any other medium from which a computer can read. Instructions may further be transmitted or received using a transmission medium. The term “transmission medium” may include any tangible or intangible medium that is capable of storing, encoding or carrying instructions for execution by the machine, and includes digital or analog communications signals or other intangible medium to facilitate communication of such instructions. Transmission media includes coaxial cables, copper wire, and fiber optics, including wires that comprise bus 1202 for transmitting a computer data signal.

In some examples, execution of the sequences of instructions may be performed by computing platform 1200. According to some examples, computing platform 1200 can be coupled by communication link 1221 (e.g., LAN, PSTN, or wireless network) to another processor to perform the sequence of instructions in coordination with one another. Computing platform 1200 may transmit and receive messages, data, and instructions, including program, i.e., application code, through communication link 1221 and communication interface 1213. Received program code may be executed by processor 1204 as it is received, and/or stored in memory 1206, or other non-volatile storage for later execution.

In the example shown, system memory 1206 can include various modules that include executable instructions to implement functionalities described herein, in the example shown, system memory 1206 includes an affective state prediction module 1230 configured to determine an affective state of a user. According to some embodiments, system memory 1206 can also include an activity-related module 1232 to ascertain activity-related data. Also, memory 1206 can include data representing physiological state analyzer module 1256, data representing stressor analyzer module 1258 and data representing stressor analyzer module 1259.

Referring back to FIG. 1 and subsequent figures, a wearable device, such as wearable device 110a, can be in communication (e.g., wired or wirelessly) with a mobile device 113, such as a mobile phone or computing device. In some cases, mobile device 113, or any networked computing device (not shown) in communication with wearable device 110a or mobile device 113, can provide at least some of the structures and/or functions of any of the features described herein. As depicted in FIG. 1 and subsequent figures, the structures and/or functions of any of the above-described features can be implemented in software, hardware, firmware, circuitry, or any combination thereof. Note that the structures and constituent elements above, as well as their functionality, may be aggregated or combined with one or more other structures or elements. Alternatively, the elements and their functionality may be subdivided into constituent sub-elements, if any. As software, at least some of the above-described techniques may be implemented using various types of programming or formatting languages, frameworks, syntax, applications, protocols, objects, or techniques. For example, at least one of the elements depicted in FIG. 1 (or any subsequent figure) can represent one or more algorithms. Or, at least one of the elements can represent a portion of logic including a portion of hardware configured to provide constituent structures and/or functionalities.

For example, affective state prediction unit 120 and any of its one or more components, such as physiological state analyzer 422 of FIG. 4, stressor analyzer 424 of FIG. 4, and/or mood formation module 423 of FIG. 4, can be implemented in one or more computing devices (i.e., any mobile computing device, such as a wearable device or mobile phone, whether worn or carried) that include one or more processors configured to execute one or more algorithms in memory. Thus, at least some of the elements in FIG. 1 (or any subsequent figure) can represent one or more algorithms. Or, at least one of the elements can represent a portion of logic including a portion of hardware configured to provide constituent structures and/or functionalities. These can be varied and are not limited to the examples or descriptions provided.

As hardware and/or firmware, the above-described structures and techniques can be implemented using various types of programming or integrated circuit design languages, including hardware description languages, such as any register transfer language (“RTL”) configured to design field-programmable gate arrays (“FPGAs”), application-specific integrated circuits (“ASICs”), multi-chip modules, or any other type of integrated circuit. For example, physiological state analyzer 422 of FIG. 4, stressor analyzer 424 of FIG. 4, and/or mood formation module 423 of FIG. 4, can be implemented in one or more computing devices that include one or more circuits. Thus, at least one of the elements in FIG. 1 or 4 (or any other figure) can represent one or more components of hardware. Or, at least one of the elements can represent a portion of logic including a portion of circuit configured to provide constituent structures and/or functionalities.

According to some embodiments, the term “circuit” can refer, for example, to any system including a number of components through which current flows to perform one or more functions, the components including discrete and complex components. Examples of discrete components include transistors, resistors, capacitors, inductors, diodes, and the like, and examples of complex components include memory, processors, analog circuits, digital circuits, and the like, including field-programmable gate arrays (“FPGAs”), application-specific integrated circuits (“ASICs”). Therefore, a circuit can include a system of electronic components and logic components (e.g., logic configured to execute instructions, such that a group of executable instructions of an algorithm, for example, and, thus, is a component of a circuit). According to some embodiments, the term “module” can refer, for example, to an algorithm or a portion thereof, and/or logic implemented in either hardware circuitry or software, or a combination thereof (i.e., a module can be implemented as a circuit). In some embodiments, algorithms and/or the memory in which the algorithms are stored are “components” of a circuit. Thus, the term “circuit” can also refer, for example, to a system of components, including algorithms. These can be varied and are not limited to the examples or descriptions provided.

In at least some examples, the structures and/or functions of any of the above-described features can be implemented in software, hardware, firmware, circuitry, or a combination thereof. Note that the structures and constituent elements above, as well as their functionality, may be aggregated with one or more other structures or elements. Alternatively, the elements and their functionality may be subdivided into constituent sub-elements, if any. As software, the above-described techniques may be implemented using various types of programming or formatting languages, frameworks, syntax, applications, protocols, objects, or techniques. As hardware and/or firmware, the above-described techniques may be implemented using various types of programming or integrated circuit design languages, including hardware description languages, such as any register transfer language (“RTL”) configured to design field-programmable gate arrays (“FPGAs”), application-specific integrated circuits (“ASICs”), or any other type of integrated circuit. These can be varied and are not limited to the examples or descriptions provided.

Although the foregoing examples have been described in some detail for purposes of clarity of understanding, the above-described inventive techniques are not limited to the details provided. There are many alternative ways of implementing the above-described invention techniques. The disclosed examples are illustrative and not restrictive.

1. A method comprising:
receiving sensor signals including data representing physiological characteristics associated with a wearable device, the wearable device being configured to receive the sensor signals from a distal portion of a limb at which the wearable device is disposed;
calculating a portion of an intensity associated with an affective state for each of the physiological characteristics in a subset of the physiological characteristics;
forming an intensity value based on the portions of the intensity;
determining a polarity value of the intensity value;
determining the affective state at a processor, the affective state being a function of the intensity value and the polarity value of the intensity value; and
transmitting data representing the affective state associated with the wearable device based on sensors configured to be disposed at the distal portion of the limb.

2. The method of claim 1, wherein forming the intensity value comprises:
aggregating the portions of the intensity to form the intensity value as an aggregated sensor-derived value.

3. The method of claim 1, wherein determining the polarity value comprises:
determining either a positive value or a negative value for the intensity value.

4. The method of claim 3, wherein determining either the positive value or the negative value for the intensity value comprises:
determining the positive value or the negative value based on the value of a heart-related physiological characteristic.

5. The method of claim 4, wherein determining the positive value or the negative value based on the value of the heart-related physiological characteristic comprises:
determining a value indicating a heart rate variability (“HRV”).

6. The method of claim 3, wherein determining either the positive value or the negative value for the intensity value comprises:
determining a value of a stress score that indicative of either the positive value or the negative value for the intensity value; and
identifying the polarity of the intensity based on the value of the stress score.

7. The method of claim, wherein determining the value of the stress score comprises:
identifying data representing activity-related score data for which the user is or has been engaged; and
calculating the polarity as a function of the activity-related score data.

8. The method of claim 1, wherein receiving the sensor signals comprises: receiving environmental sensor data.

9. The method of claim 1, wherein receiving the sensor signal comprises: receiving a bio-impedance signal from the distal end of the limb at which the wearable device is disposed.

10. The method of claim 1, wherein receiving the sensor signal comprises: receiving the data representing the physiological characteristics including one or more of a heart rate, a respiration rate, and a Mayer wave rate.

11. An apparatus comprising:
a wearable housing configured to couple to a portion of a limb at its distal end;
a subset of physiological sensors configured to provide data representing physiological characteristics; and
a processor configured to execute instructions to implement an affective state prediction unit configured to:
calculate a portion of an intensity associated with an affective state for each of the physiological characteristics in a subset of the physiological characteristics;
form an intensity value based on the portions of the intensity;
determine a polarity value of the intensity value;
determine the affective state as a function of the intensity value and the polarity value of the intensity value; and
transmit data representing the affective state associated with the subset of physiological sensors configured to be disposed at the distal portion of the limb.

12. The apparatus of claim 11, wherein the affective state is associated with an approximated emotional physiological state of a wearer around which the wearable housing is disposed.

13. The apparatus of claim 11, wherein the processor further is configured to execute instructions to:
determine a value of a physiological characteristic; and
determine the polarity of the intensity as either positive or negative based on the value of the physiological characteristic.

14. The apparatus of claim 13, wherein the processor further is configured to execute instructions to:
determine the affective state based on a value for one of a negative high-intensity physiological state, a negative low-intensity physiological state, a positive high-intensity physiological state, and a positive low-intensity physiological state.

15. The apparatus of claim 11, wherein the processor further is configured to execute instructions to:
analyze activity-related data to determine whether the intensity is of a level within a range of negative affectivity or within a range of positive affectivity.

16. The apparatus of claim 11, wherein the processor further is configured to execute instructions to:
establish communication with an environment controller configured to modify an environmental factor of an environment in which a wearer of the wearable device is located; and
transmit the data representing the affective state to the environment controller to adjust the environment factor.

17. The apparatus of claim 16, wherein the processor further is configured to execute instructions to:
cause the environmental controller to modify operation of one or more of an auditory source, a visual source, and a heating ventilation and air conditioning (“HVAC”) source to modify a sound, a light, and a temperature, respectively, as the environmental factor.

18. The apparatus of claim 11, wherein the processor further is configured to execute instructions to:
establish communication with a social networking service platform configured to generate a presentation of the data representing the affective state on a web site; and
transmit the data representing the affective state to the social networking service platform to publish the affective state associated with a wearer of the wearable device.

19. The apparatus of claim 11, wherein the processor further is configured to execute instructions to:
establish communication with a computing device associated with a person co-located with a wearer of the wearable device; and
transmit the data representing the affective state to the computing device associated with the person to provide feedback to the person as to a social interaction between the person and the wearer.

20. The apparatus of claim 19, wherein the processor further is configured to execute instructions to:
present a recommendation to the person via a display on the computing device to modify the social interaction to urge the data representing the affective state to an increased positive intensity value.
